(19) United States
(12) Patent Application Publication
(10) Pub. No.: US 2009/0232832 A1
(43) Pub. Date: Sep. 17, 2009

(54) Polyclonal antiserum against a universal tumor antigen

(75) Inventors: Álvaro Joaquin Luongo Céspedes, Montevideo (UY); Julio José Battistoni Spinelli, Montevideo (UY); Alvaro Luis Lamas Alfonsin, Montevideo (UY)

Correspondence Address: FOLEY AND LARDNER LLP;SUITE 500, 3000 K STREET NW, WASHINGTON, DC, 20007, US

(73) Assignee: WIDNES COMPANY INC

(21) Appl. No.: 11/921,167

(22) PCT Filed: Jun. 2, 2006
(86) PCT No.: PCT/EP2006/005269

§ 371 Date: Dec. 19, 2007

(30) Foreign Application Priority Data
Jun. 3, 2005 (EP) 05012053.4

(51) Int. Cl. A61K 39/395; A61P 35/00

(52) U.S. Cl. 424/172.1; 424/178.1

(57) ABSTRACT

The present invention relates to a polyclonal antiserum against a universal tumor antigen obtainable by (i) eliciting an in vivo humoral response against embryonic tissue in a non-human vertebrate, whereby said embryonic tissue is of the same genetic line as the non-human vertebrate; (ii) recovering from the immunized non-human animal spleen and isolating from said spleen individual spleen cells/lymphocytes; (iii) eliciting a second in vivo humoral response to the isolated spleen cells/lymphocytes suspension obtained in step (ii) in a further non-human animal of the same genetic line as the non-human animal of step (i); and (iv) isolating the desired polyclonal antiserum from said animal. Furthermore, the invention provides for the use of the polyclonal antiserum for the preparation of a pharmaceutical composition for the amelioration, prevention and/or treatment of cancer, in particular cancer of the breast, lung, prostate, uterus, colon, stomach or bladder. Additionally the invention relates to the use of the polyclonal serum of the invention for the preparation of a pharmaceutical composition, wherein said pharmaceutical composition is to be administered to a subject in need of treatment in combination with a further anti-proliferative drug or medicament.

The present invention relates to a polyclonal antiserum against a universal tumor antigen obtainable by (i) eliciting an in vivo humoral response against embryonic tissue in a non-human vertebrate, whereby said embryonic tissue is of the same genetic line as the non-human vertebrate; (ii) recovering from the immunized non-human animal spleen and isolating from said spleen individual spleen cells/lymphocytes; (iii) eliciting a second in vivo humoral response to the isolated spleen cells/lymphocytes suspension obtained in step (ii) in a further non-human animal of the same genetic line as the non-human animal of step (i); and (iv) isolating the desired polyclonal antiserum from said animal. Furthermore, the invention provides for the use of the polyclonal antiserum for the preparation of a pharmaceutical composition for the amelioration, prevention and/or treatment of cancer, in particular cancer of the breast, lung, prostate, uterus, colon, stomach or bladder. Additionally the invention relates to the use of the polyclonal serum of the invention for the preparation of a pharmaceutical composition, wherein said pharmaceutical composition is to be administered to a subject in need of treatment in combination with a further anti-proliferative drug or medicament.

About 1 million new cases of cancer have been diagnosed in the United States in 2003, and about half a million people will die of the disease. Cancer is thus the second leading cause of death in the US and worldwide (American Cancer Society, 2003; Stern, 2004). Conventional methods of cancer treatment like chemotherapy, surgery or radiation therapy, are mostly limited in their efficacy since they are often unspecific and inaccurate (Kimball, 1993). In many cases tumors, however, specifically express genes whose products are required for inducing or maintaining the malignant state. These proteins may serve as antigen markers for the development and establishment of efficient anti-cancer treatments. Antigens are known to elicit an immune response. These antigens are either proteins, polysaccharides, lipids, or glycolipids, which are recognized as “foreign” by lymphocytes, i.e. B and T cells. Exposure of lymphocytes to an antigen as described above elicits a rapid cell division and differentiation response resulting in the formation of clones of the exposed lymphocytes. B cells produce plasma cells which in turn produce antibodies which selectively bind to the antigens. Whereas a monoclonal antibody is the product of a single clone of B lymphocytes, polyclonal antibodies are produced by several clones of B lymphocytes.

Polyclonal antibodies have successfully been used to induce apoptosis and to block support of tumor growth in mice by vaccinating rabbits with murine endothelial cells. This approach yielded polyclonal immunoglobulin with potent antiangiogenic activity. The polyclonal antibody showed antitumor activity in mouse tumor models and demonstrated utility in radioimaging of tumors in vivo (Scappaticci, 2003). Okaji et al. (2004) also report on an inhibition effect on angiogenesis and metastasis of colon cancer through autoimmunity. BALB/c mice were immunized in this study with hepatic sinusoidal endothelial cells of Colon-26 cancer which induced both preventive and therapeutic anti-tumor immunity that significantly inhibited the development of metastases. The immunoglobulin response involved IgM and IgG subclasses of antibodies. Furthermore, patent application WO 01/89563 envisages and describes the use of purified polyclonal antibodies for treating allergy. In particular, it is disclosed that in the case of allergens which are often complex proteins, a treatment with polyclonal antibodies is clearly advantageous and preferable to the use of monoclonal antibodies.

According to Abelev, four groups of antigens can be differentiated: (i) viral tumor antigens which are identical for any viral tumor of this type, (ii) carcinogenic tumor antigens which are specific for patients and for the tumors, (iii) isoantigens of the transplantation type or tumor-specific transplantation antigens which are different in all individual types of tumor but are the same in different tumors caused by the same virus; and (iv) embryonic antigens. During carcinogenesis cells are dedifferentiated and may thus acquire an embryonic state of gene expression. Accordingly, embryonic antigens which are specific to embryonic development of an organism can be found within these cancerous cells. Such antigens can immunize the organism against tumors and can be used for the establishment of anti-cancer treatments. The most prominent embryonic antigens are α-fetoprotein and carcinogenic embryonic antigen (CEA). α-fetoprotein is a major transport protein in the fetus and serves, inter alia, as a serum marker in the clinical laboratory for cancer and for fetal defects (Tatarinov, 1965; Abelev, 1971; Mizejewski, 2003) and as target for specific immunotherapy against head and neck cancer (Kass, 2002). CEA is a glycoprotein and belongs to the immunoglobulin superfamily and has similarity to the intercellular adhesion molecule 1 (ICAM-1). CEA was first identified as an antigen that was present in both fetal colon and colon adenocarcinoma but that was absent from healthy adult colon. CEA is one of the most widely used markers for carcinomas of pancreas, stomach, and breast and the most frequently used marker in colorectal cancer. CEA also serves as therapeutic target for, e.g. specific antibody compositions.

Although the above characterized embryonic antigens have become pivotal in the detection and the monitoring of cancer, there is still significant lack of information and comprehension concerning the presence of embryonic antigens on tumor cells. WO 99/53952 provides for a method for the production of a specific antiserum against universal tumorous antigens which are differentially derived from embryonic tissue. The corresponding antiserum was proposed as diagnostic tool for the detection of (malignant) tumors.

The prior art has described several monoclonal antibodies directed against specific cancer associated epitopes. Examples are, inter alia, monoclonal antibodies directed against HER2/c-erb-B2 employed in cancer treatment of the mama. Monoclonal antibodies used as anti-human breast cancer antibodies have, inter alia, be described in EP-A2 0 153 114 or WO 89/06692. However, the use of monoclonal antibodies may also be described as sub-optimal associated with certain disadvantages. This is due to the fact that monoclonal antibodies are directed against very specific single antigenic epitopes. Thus, if the target is of a complex nature, not yet molecularly identified or if not only one target, but rather a group of different targets is involved, a monoclonal antibody would either recognize said target with a very low avidity or not at all and could hence only be used in a sub-optimal manner. A single monoclonal antibody can, in consequence, not be expected to exhaustively cover more than a minority of the possibly relevant epitopes on a cancer cell, as is also illustrated, for example, by immunological tests which need to use more than one monoclonal antibody in order to obtain a clear signal.

Accordingly, there is a need in the art for therapeutic means and methods which provide for an efficient amelioration, prevention and/or treatment of proliferative disorders, in particular, cancer.

Thus, the technical problem underlying the present invention is to comply with the needs described above and to provide for a treatment of a multitude of different cancer types or simultaneously.

Accordingly, the present invention relates to a pharmaceutical composition comprising a polyclonal antiserum obtained by the following steps:

(i) eliciting an in vivo humoral response against non-human embryonic tissue in a non-human vertebrate, whereby said embryonic tissue is of the same genetic line as the non-human vertebrate; (ii) recovering from the immunized non-human animal spleen and isolating from said spleen individual spleen cells/lymphocytes; (iii) eliciting a second in vivo humoral response to the isolated spleen cells/lymphocytes suspension obtained in step (ii) in a further non-human animal of the same genetic line as the non-human animal of step (i); and (iv) isolating the desired polyclonal antiserum from said animal.

The term “polyclonal antiserum” as used in the present invention relates to polyclonal or multispecific antibodies, Fab fragments, F(ab′)2 fragments, anti-idiotypic antibodies and epitope-binding fragments of any of the above. Said polyclonal or multispecific antibodies, Fab fragments, F(ab′)2 fragments, anti-idiotypic antibodies and epitope-binding fragments may be comprised in an obtained serum as isolated in step (iv) as outlined herein above or may be further purified corresponding methods are known in the art and, inter alia, described in Harlow and Lane “Antibodies” (1988). The term “antibody”, as used herein, refers to immunoglobulin molecules, i.e. molecules that contain an antigen binding site that immunospecifically binds an antigen. The immunoglobulin molecules of the invention can be of any class (e.g. IgG, IgE, IgM, IgD, IgA and IgY), or subclass (e.g. (IgG1, IgG2, IgG3, IgG4, IgA1 and IgA2) of immunoglobulin molecule.

The term “eliciting an in vivo humoral response in a non-human vertebrate” relates to the provocation of an immune response in a non-human vertebrate, in particular the provocation of an antibody response to/against embryonic tissue of a non-human vertebrate or a purified fraction thereof. Said antibody response comprises primary as well as secondary antibody responses to the antigenic challenge with said embryonic tissue thereof. The term “eliciting an in vivo humoral response”, accordingly, relates to the provocation of an immune reaction involving the production of antibodies directed towards a plurality of antigens comprised in said embryonic tissue. As illustrated in the appended examples, the non-human preferably non-human vertebrate embryonic tissue may in particular be tissue and cells obtained from homogenized mouse embryos, for example d12 embryos. Accordingly, the term “non-human embryonic tissue” as employed herein above (in step (i)) comprises in a most preferred embodiment isolated cells obtained from homogenized embryos (or embryonic tissues). Most preferably the “embryonic tissue cells” to be employed do not comprise any tissue clumps. Corresponding further details may be obtained from the appended non-limiting examples and in particular in example 1. As illustrated therein, in a preferred embodiment of the invention the “embryonic tissue”/“embryonic tissue cells” are derived from mouse, most preferably a CBA/CaJ mouse. Preferably, corresponding embryonic cells for immunization are derived from 10 to 14 d old mouse embryos, most preferably 12 d old mouse embryos.

The term “genetic line” relates to the fact that, in accordance with this invention, the embryonic tissue/the embryonic cell to be used to elicit the immunological response in said non-human vertebrate are at least of the same species. Accordingly, if embryonic tissue from a mouse is employed the corresponding immunological response of step (i) is to be elicited in a mouse. Most preferably the term “same genetic line” corresponds to the fact that animals are used as source for the embryonic tissue which are of the same or are in the same genetic background as the animal where the corresponding immune response is elicited. As for example, illustrated in the appended examples, it is envisaged and exemplified that CBA/CaJ mice are employed as a source for the non-human embryonic tissue and that also mice of the same strain (CBA/CaJ) are employed for immunization with the isolated embryonic tissue.

In a preferred embodiment of the pharmaceutical composition the non-human animal to be immunized is a mouse and in a most preferred embodiment the mouse is a mouse of the CBA strain. Mice of the CBA strain are known in the art and are obtainable from e.g. Jackson Laboratory, USA. The CBA strain comprises, inter alia, CBA/H, CBA/J, CBA/CaJ and CBA/N mice. As shown in the appended examples, most preferred are CBA/CaJ mice, inter alia, obtainable from Jackson Laboratories under stock number 000654. However, the “non-human vertebrate” mentioned herein above may be selected from the group consisting of rat, rabbit, chicken, sheep, horse, goat, pig and donkey. Yet, most preferably said vertebrate is a mouse.

A further preferred embodiment of the present invention relates to a pharmaceutical composition or a use wherein the polyclonal antiserum is a purified polyclonal antiserum.

The term “purified polyclonal antiserum” relates to (an) isolated polyclonal antibody(ies) or fragment(s) thereof which has been purified via standard methods known in the art. However, said term also relates to “immuno”-purified antibody preparations whereby the obtained polyclonal antibody serum of step (iv) of the method described herein is contacted with non-embryonic (i.e. post-natal) organs, organ lysates or cells derived from non-embryonic organs or organ lysates. Preferably, said non-embryonic (i.e. post-natal) organs, organ lysates or cells derived from non-embryonic organs or organ lysates are derived from an non-human animal of the same species as the animal wherein said in vivo tumoral response is/was elicited. Such a purification can be by any mixture, absorbance or incubation procedure suitable to avoid undesirable cross reactivity known to the person skilled in the art. Preferably, the purification procedure carried out in accordance with the procedure as described in Example 3.

The term “purified polyclonal serum” specifically relates to an isolated polyclonal antibody or fragment thereof, which has been purified to homogeneity, in particular, it has been purified to a purity level of at least 95%, more preferably of at least 96%, even more preferably of at least 97%, particularly preferred of at least 98% and most preferably of at least 99% purity. The purity of the polyclonal antiserum may be confirmed by methods known in the art and most preferably as described in the appended examples. Accordingly, the purified polyclonal antiserum preparation of the invention comprises preferably less than 5% contaminating, unrelated proteins or protein fragments. Most preferably, said preparation comprises less than 2% contaminating, unrelated proteins or protein fragments.

Preferably the polyclonal antiserum comprises a fraction of the antiserum, like the fraction comprising immunoglobulins.

Most preferably and advantageously, the fraction of the polyclonal antiserum is an IgG fraction or is or comprises a F(ab′)₂ fragment fraction.

Various alternative methods exist for the fractionation of the polyclonal antiserum and are known to the person skilled in the art. One convenient technique involves the use of affinity chromatography utilising, for example, Protein G or Protein A columns. Typically columns contain 5 ml or 10 ml of packed protein A or G agarose. The size of the column is determined by the binding capacity of protein A/G and the amount of antiserum that must be processed. Protein A and protein G bind about 20 mg of IgG per ml of conjugated agarose.

The term “Fab fragment” refers to an antibody fragment which contains the antigen-binding activity. It corresponds to the two identical arms of the antibody molecule, which contain the complete light chains paired with the V_{H} and C_{H}1 domains of the heavy chains. It is a disulfide-linked heterodimer, each chain of which contains one immunoglobulin C domain and one V domain wherein the juxtaposition of the V domains forms the antigen-binding site. The term “F(ab′)₂ fragment” refers to an antibody fragment in which the two antigen-binding arms of the antibody molecule remain linked. In this case the remaining part of the heavy chain is cut into several small fragments. The Fab and F(ab′)₂ fragments have exactly the same antigen-binding specificity as the original antibody but are unable to interact with any effector molecule or cell.

Several approaches in the production of antibody fractions, antibody preparations and/or antibody fragments (starting from obtained sera) have been reported in the literature. For example, U.S. Pat. No. 4,849,352 describes the production of both Fab fragments through the digestion of antibodies with immobilized papain and subsequently purifying the fragments through immunoaffinity. It also describes the production of F(ab′)₂ fragments through the digestion of antibodies with immobilized pepsin, obtaining F(ab′)₂ and Fc small fragments and subsequently purifying the immunoglobuline fragments through gel filtration. Another approach is shown in U.S. Pat. No. 5,733,742 in which a process to produce Fab fragments using whole blood in a sterile medium is described, in which the whole blood is put directly into contact with the enzyme, free or immobilized, that has preferably been purified. Subsequently, the cell residues are removed by centrifugation, separating and recovering the resulting fragments that are subsequently purified preferably by immunoaffinity. An additional approach to the production of Fab fragments is shown in U.S. Pat. No. 4,814,433 which describes a procedure for obtaining papain free Fab. Subsequently, the fragments are purified by passing the solution along a column with protein A in which the Fc fragments and the hybrid compounds were retained. Further methods involve the digestion of pepsin and the precipitation of the fraction of the fragments with ammonium or sodium sulfates, but a pre-separation is usually done with the antibodies by precipitation with sulfate and then digestion of the antibody fraction.

The most important clinical use of a polyclonal antiserum as obtained by the method as characterized herein above is in the preparation of a pharmaceutical composition for the amelioration, prevention and/or treatment of cancer. Accordingly, the polyclonal antiserum may also be used in a method of treatment and/or amelioration of cancer in a subject. In a preferred embodiment the subject is a human patient.

Hence, one aspect of the invention is the use of polyclonal serum for the amelioration, prevention or treatment of a subject with cancer, wherein the cancer tumor or cancer cells of said subject express an antigen bound by the polyclonal antiserum of the invention. The treatment involves administering, inter alia, an amount of polyclonal antibodies to said subject that is sufficient to have a therapeutic effect on said subject. Another aspect of the invention is the administration of a polyclonal antibody conjugated with a label. The artisan is familiar with the type of label that can be used on antibodies, such as chemotherapeutic agents, apoptotic agents, agents which inhibit DNA expression, or radioactive agents. Of the many therapeutic agents known in the art, therapeutic agents selected from the group consisting of radioisotopes, inflammatogenic agents, enzymes, antisense molecules, peptides, cytokines, and chemotherapeutic agents are preferred.

Thus, in a further aspect, the polyclonal antibody/polyclonal serum or fragment(s) thereof according to the invention may be administered to the patient in need thereof, wherein the antibody protein or fragment thereof comprised in said serum or obtained antibody (fragment) preparation is conjugated to a therapeutic agent. Procedures for conjugating an antibody with chemotherapeutic agents have been previously described. Suitable chemotherapeutic agents are known to those skilled in the art and include, but are not limited to daunomycin, adriamycin, etoposide, cyclophosphamide, methotrexate, vindesine, neocarzinostatin, cisplatin, chlorambucil, cytosine arabinoside, 5-fluorouridine, melphalan, ricin, abrin and calicheamicin.

These agents can be chemically conjugated to the polyclonal antibody of the present invention or fragment thereof via chemical crosslinking by any of a variety of well-known chemical procedures, such as the use of bifunctional or hetero-bifunctional cross-linkers, e.g. SPDP, 2-iminothiolane, carbodiimide or glutaraldehyde.

Procedures for conjugating e.g. chlorambucil with antibodies or antibody fragments are described by Flechner, 1993; Ghose, 1972; and Szekerke, 1972. Procedures for conjugating e.g. daunomycin and adriamycin to antibodies are described by Hurwitz, 1975 and Arnon, 1982. Procedures for preparing, e.g. antibody-ricin conjugates are described in U.S. Pat. No. 4,414,148 and by Osawa, 1982. Procedures for conjugating calicheamicin to antibodies can be found in Hamann, 2002. Procedures for conjugation of doxorubicin to antibodies can be derived, e.g. from Stan, 1999. Further guidance for the production of various immunotoxins can be found, for example, in Thorpe, 1982; Waldmann, 1991, Blakely, 1998; Waldmann, 1988 or Cumber, 1985.

Chemical binding of polypeptides like ricin or abrin to one of the aforementioned antibodies or antibody fragments may be carried out through methods known in the art, like chemical crosslinking with bifunctional or heterobifunctional reagents (examples: glutaraldheyde or N sucinimydil 3-(2-pyridilthio) propionate, SPDP); see Waldmann, 1988 and Cumber, 1985.

Bispecific antibodies have also been proposed in the therapy of cancer, which are formed by bonding together two variable domains of antibodies, each one specific for a different epitope. Thus, bispecific antibodies are hybrid immunoglobulins bearing two different antigen-binding sites (paratopes) that can be prepared, e.g. by chemical linkage (Brennan, 1985). Cancer therapeutic bispecific antibodies are formed by one paratope directed to a tumor antigen while the other is directed to cell-surface molecule capable of mediating phagocytic or lytic responses by macrophages, natural killer cells, T-cells, or other effector cells (Van Spriel, 2000; Kipriyanov, 2004; Fanger 1991; Davol, 2004).

Further therapeutic agents to be conjugate to the polyclonal antibody envisaged by the present invention are radioisotopes or agents containing radioisotopes. Among the radioisotopes, gamma, beta-, and alpha-emitting radioisotopes may be used. beta-emitting radioisotopes are preferred as therapeutic radioisotopes. Among the type of such radiolabels which can be used are ¹¹¹Indium, ¹³¹Iodine, ¹²⁵Iodine, ⁹⁰Yttrium, ¹⁷⁷Lutetium, ⁸⁶Rhenium, ⁸⁸Rhenium, ²¹³Bismuth, various isotypes of cobalt, indium, and other radioactive materials. See, e.g., WO 93/05804 for protocols for radiolabeling antibodies. ¹⁸⁶Rhenium, ¹⁸⁸Rhenium, ¹³¹Iodine and ⁹⁰Yttrium have been shown to be particularly useful [beta]-emitting isotopes to achieve localized irradiation and destruction of malignant tumor cells. Therefore, radioisotopes selected from the group consisting of ¹⁸⁶Rhenium, ¹⁸⁸Rhenium, ¹³¹Iodine and ⁹⁰Yttrium are particularly preferred as therapeutic agents conjugated to the proteins of the polyclonal antiserum of the invention.

Accordingly, radionucleotides radiotherapy research has focused on the use of radiolabeled antibodies to deliver doses of radiation directly to the cancer cells (radioimmunotherapy). The antibody(ies), antibody fragment(s) obtained in accordance with this invention may be chemically bond to radionucleotides by methods known in the art, see, inter alia, Liu Yuanfang, 1991; Eckelman, 1980.

According to the present invention the tumor specific polyclonal antiserum may trigger various “killing mechanisms” for tumors or tumor cells. For example, the complement system may be activated onto the tumor cell. Whole immunoglobulin molecules, i.e. polyclonal antibodies, are able to locate tumor cells and activate the complement system resulting in the formation of a membrane pore and cell lysis. (Harris 2004).

In an alternative, immune effector cells could be summoned towards the tumor growing site and cytotoxic properties could be activated in situ. In particular, immunoglobulin molecules can recruit and activate effectors cells resulting in tumor cell death by antibody dependent cell-mediated cytotoxicity (ADCC). Natural killer cells (NK) carry receptors for the Fc fragment of IgG; such an attachment activates a cascade of events that conclude in the damage of the cell membrane inducing the apoptosis of the target cell.

Furthermore, cytotoxic molecules or atoms which compromise the cell behaviour or the cell viability like toxins or radionucleids as described herein above may be disposed at the location of the tumor or the tumor cells. Such a specific targeting of cytotoxic agents to tumor cells has the potential to reach high concentrations at tumor sites, without the dose limiting side-effects of systemic administration. Once attached to the cell surface, the conjugate is engulfed into the cell cytoplasm where cell enzymes cut the drugs or toxins free from the antibodies. Upon release, the drugs or toxins damage the cell irreversibly inducing cell death. For instance, radio-labeled antibodies bound to tumor cells concentrate the radioactivity onto the cells. Irradiation at so short distances from the nucleus, induce cancer cell death. (Goldenberg, 1993).

Bispecific immunoglobulins therapy is based on the selective recruitment of an immune effector mechanism towards a defined disease-related target structure. Thus, bispecific molecules serve as a linkage between an effector mechanism and its target. A plethora of effector mechanisms can be envisaged for therapeutic applications and a large number have already been evaluated. These include the recruitment of effectors molecules (e.g. toxins, drugs, prodrugs, cytokines, radionuclides), the retargeting of effector cells (e.g. cytotoxic T lymphocytes, NK cells, macrophages, granulocytes) and the retargeting of carrier systems.

The polyclonal serum and/or the obtained antibodies/antibody of this invention is (are) particularly useful in the medical intervention of proliferative disorders, cancer and/or in tumor therapy.

The terms “cancer” or “tumor” as used in the context of the present invention includes any disease associated with malignant growth. Cancer or tumor according to the invention includes, but is not limited to:

1) Cancers of the head and neck, including (i) cancers of the nasal cavity and paranasal sinuses, cancer of the nasopharynx cancer of the oral cavity and cancers of oropharynx, tumors of the Larynx and hypopharynx, tumors of the salivary glands and paragangliomas as well as (ii) variants of squamous cell carcinomas, verrucous carcinomas, sarcomatoid squamous cell carcinomas, lymphoepitheliomas, adenoid cystic carcinomas, mucoepidermoid carcinomas, acinic cell carcinomas, adenocarcinomas and neuroendocrine tumors.

2) Cancers of the lung, including (i) non-small-cell-lung cancers which includes carcinoma, adenocarcinoma, large cell (undifferentiated) carcinomas and squamous cell carcinomas as well as (ii) small cell lung cancers including the oat cell cancers, lymphocyte-like cancers, the intermediate cancers, and SCLC (combined with squamous cancer or adenocarcinoma).

3) Neoplasms of the mediastinum, including (i) neoplasms of the anterosuperior mediastinum which comprise thymic neoplasms, lymphomas, germ cell tumors, and carcinoma, Brachial, enteric, and pericardial cysts, aberrant parathyroid tumors and thyroid neoplasms, (ii) middle mediastinum neoplasms which include cystic lesions lymphomas, mesenchymal tumors and carcinoma as well as (iii) posterior mediastinum neoplasms, which include neurogenic tumors, cysts lesions, mesenchymal tumors, and endocrine neoplasms.

4) Cancers of the gastrointestinal tract, including (i) cancer of the esophagus, cancer of the stomach, cancer of the pancreas, hepatobiliary cancers, cancer of the small Intestine, cancer of the colon, cancer of the rectum, cancer of the anal region as well as squamous cell carcinoma, adenoacanthoma, carcinoid tumors, leiomyosarcoma, mesenchymal tumors epithelial neoplasms, mixed HCC/cholangiocarcinomas, lymphomas and melanomas.

5) Cancers of the genitourinary system, including cancer of the kidney and ureter, cancer of the bladder, cancer of the prostate and cancer of the urethra and penis as well as transitional cell carcinomas, adenocarcinomas, squamous cell carcinoma melanoma, basal cell carcinoma and mesenchymal tumors.

6) Cancers of the testis, including carcinoma in situ, seminoma and nonseminomatous, leydig cell tumors, sertoli cell tumors, granulosa cell tumors, gonadoblastomas, mesotheliomas, sarcomas, adenocarcinomas of the rete testis, epidermoid cyst, lymphomas and metastatic carcinomas.

7) Gynecologic cancers, including endometrial cancer, cancer of the cervix, vagina and vulva, cancer of the uterine body, gestational trophoblastic diseases, ovarian cancer, Fallopian tube carcinoma and peritoneal carcinoma as well as adenocarcinoma in situ, squamous intraepithelial carcinomas, malignant mixed Mullerian tumor (MMT), trophoblastic tumors and germ or stromal cells tumors.

8) Cancer of the breast, i.e. malignant tumors of the breast including carcinomas in situ, ductal carcinomas in situ and lobular carcinomas in situ.

9) Cancers of the endocrine system selected from the group consisting of thyroid tumors, parathyroid tumors, adrenal tumors, pancreatic endocrine tumors, carcinoid tumors and the carcinoid syndrome, multiple endocrine neoplasia type 1 (MEN 1), including carcinomas and adenomas.

10) Sarcomas of the soft tissues and bone including (i) soft tissue sarcoma, (ii) sarcomas of the bone comprising malignant spindle cell tumors, parosteal osteosarcomas, periosteal osteosarcomas, Paget's sarcomas, High-Grade surface osteosarcomas and small cell osteosarcomas, giant cell tumors of bone, giant cell tumors of the sacrum, malignant fibrous histiocytomas, fibrosarcomas of bone, malignant hemangioendotheliomas of bone, chordomas, small round cell sarcomas of bone and lymphomas of bone (diffuse large cell lymphomas).

11) Malignant mesotheliomas, including epithelial, sarcomatoid, and mixed tumors.

12) Cancers of the skin selected from the group consisting of basal cell carcinomas, squamous cell carcinomas, cancer-associated genodermatoses including Xeroderma pigmentosum, nevoid basal cell carcinoma syndrome, familial dysplastic nevus syndrome, multiple self-healing, epitheliomas of Ferguson-Smith, Muir-Torre syndrome, Cowden's syndrome, Gardner's syndrome and Carney's syndrome as well as tumors arising from epidermal Merkel's cell, Merkel's cell carcinomas, tumors arising from epidermal Langerhans cells, tumors of hair follicles, tumors of sebaceous glands, tumors of apocrine glands, tumors of eccrine glands, tumors arising from the dermis and lymphoreticular tumors and related conditions

13) Malignant melanomas, including cutaneous melanomas and Intraocular melanomas.

14) Neoplasms of the central nervous system, including oligoastrocytomas, choroid plexus tumors, astrocytoma-oligodendrogliomas, astrocytoma-ependymomas, astrocytomas, oligodendrogliomas, ependymomas and glioblastomas.

15) Cancers of childhood, including Wilms' tumor, neuroblastomas, rhabdomyosarcomas, retinoblastomas, Ewing's sarcomas and peripheral primitive neuroectodermal tumor, solid tumors of childhood as well as malignant gonadal and extragonadal germ cell tumors like testis tumors, ovary tumors, mediastinum tumors and vagina tumors as well as yolk sac tumors, embryonal carcinomas, seminomas, choriocarcinoma, teratomas, teratocarcinomas and dysgerminomas.

16) Lymphomas, including (i) cell non-Hodgkin's lymphomas like small lymphocytic lymphomas/B-cell, chronic lymphocytic leukemias (SLL/B-CLL), lymphoplasmacytoid lymphomas (LPL), follicular lymphomas (FL), mantle cell lymphomas (MCL), diffuse large-cell lymphomas (DLCL), and Burkitt's lymphoma (BL), (ii) AIDS-associated lymphomas, (iii) T-cell non-Hodgkin's lymphomas like anaplastic large-cell lymphomas (ALCL), cutaneous T-cell lymphomas (CTCL), and adult T-cell leukemias/lymphomas (ATLL), (iv) Hodgkin's disease, (v) leukemias including acute lymphoblastic leukemia (ALL) and acute myelogenous leukemia (AML), (vi) non-Hodgkin's lymphomas like lymphoblastic lymphomas, small noncleaved cell lymphomas, Burkitt's lymphomas and large cell lymphomas.

17) Leukemias, including acute lymphoblastic leukemias (ALL), chronic lymphocytic leukemias (CLL), prolymphocytic leukemias, hair Y cell leukemias, T-cell chronic lymphocytic leukemias, chronic myelogenous leukemias and plasma cell neoplasms.

18) AIDS-related malignancies like Karposi's sarcomas and non-Hodgkin's lymphomas.

In a most preferred embodiment, the cancer to be treated, ameliorated and/or even prevented by the use of the polyclonal antiserum of the invention is cancer of the uterus.

The invention further relates to the use of the pharmaceutical composition of the invention (comprising the inventive polyclonal antiserum as defined herein) for the treatment of cancer, wherein the pharmaceutical composition according the invention is administered once to several times to an individual in need thereof, the tumor cells are destroyed by the radioisotope linked to the antibody protein or by the chemotherapeutic agent, and the therapeutic success is monitored. The method of treating tumors as described above may be effected in vitro or in vivo. Cancer is defined as set out above. Since during prolonged therapeutic application of antibodies anaphylactic shocks may be induced due to the production of immunocomplexes the concomitant treatments of the patient with immunosuppressors as corticoids is also envisaged.

Said antibody molecule to be administered or pharmaceutical composition or medicament may further comprise a pharmaceutically acceptable carrier or excipient. By “pharmaceutically acceptable carrier” is meant a carrier that is physiologically acceptable to the administered patient. One exemplary pharmaceutically acceptable carrier is physiological saline. A pharmaceutically acceptable carrier can also contain physiologically acceptable compounds including, for example, carbohydrates, such as glucose, sucrose or dextrans, antioxidants, such as ascorbic acid or glutathione, chelating agents, low molecular weight proteins or other stabilizers or excipients. Other pharmaceutically-acceptable carriers and their formulations are well-known and generally described in, for example, Remington's Pharmaceutical Sciences, 1990. One skilled in the art would know that the choice of a pharmaceutically acceptable carrier, including a physiologically acceptable compound, depends, for example, on the route of administration of the composition.

“Administering” as used herein means providing the composition to the patient in a manner that results in the composition being inside the patient's body. Such an administration can be by any route suitable as determined by the artisan. The pharmaceutical compositions of the present invention may be applied by different routes of application known to the expert, notably intravenous injection or direct injection into target tissues. For systemic application, the intravenous, intravascular, intramuscular, intraarterial, intraperitoneal, oral, or intrathecal routes are preferred. A more local application can be effected subcutaneously, intracutaneously, intracardially, intralobally, intramedullarly, intrapulmonarily or directly in or near the tissue to be treated (connective-, bone-, muscle-, nerve-, epithelial tissue). Therapy with the polyclonal antiserum may be the entire therapeutic regime, or it may be a part of a regime which includes, e.g., chemotherapy, therapy with an additional antibody or other forms of standard therapeutic approaches to cancer.

Most preferably, the pharmaceutical composition according to the invention is to be administered intravenously.

In an animal or human body, it can prove advantageous to apply antibody or medicament as described above via an intravenous or other route, e.g. systemically, locally or topically to the tissue or organ of interest, depending on the type and origin of the tumor treated. For example, a systemic mode of action is desired when different organs or organ systems are in need of treatment as in tumors that are diffuse or difficult to localize. A local mode of action would be considered when only local manifestations of neoplastic action are expected, such as, for example local tumors. Accordingly, the pharmaceutical composition of the invention may be applied by intravenous injection or any other suitable way of administration at a location close to the cancer cells or the cancerous tissue.

Regardless of the route of administration selected, the pharmaceutical composition of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the pharmaceutical composition of the present invention employed, the route of administration, the time of administration, the rate of excretion of the pharmaceutical composition being employed, the duration of the treatment, other drugs, compounds and/or materials used in combination with the pharmaceutical composition employed, the age, sex, weight, condition, general health and prior medical history of the patient being treated, and like factors well known in the medical arts.

In certain, non-limiting embodiments of the invention, the polyclonal antiserum is administered as a 1 to 10 mg, preferably 1.5 to 5.5 mg and more preferably a 2 mg dose dissolved in 50 mL buffer solution like saline solutions. Accordingly, one administration protocol may comprise doses of 1 to 10%, preferably 2 to 6%, more preferably 4 to 5% and most preferably 4% of the antibody preparation. Preferably, said solution is infused slowly, preferably over approximately 1 to 100 minutes, preferably 10 to 60 minutes, more preferably over about 20 minutes. Yet, also longer and shorter administrations are envisaged and are within the skill of any attending person skilled in the art, for example the attending physician.

If an allergic or other reaction occurs that may limit the completion of the dose, then a lower dose may be employed at that time or with subsequent treatments, so that the expected dose range would be 1-2 mg per treatment. Premedication with oral or intravenous dyphenhydramine (25 to 50 mg) is usually administered to lessen the risk of allergic reaction to the protein. Administration of the polyclonal antiserum may be started after recovery from any required surgery that is done prior the administration and then continued up to, and during, the treatment period.

The invention also provides for the use of the polyclonal serum of the invention as described and defined herein for the preparation of a pharmaceutical composition, wherein said pharmaceutical composition is to be administered to a subject in need of treatment in combination with a further anti-proliferative drug or medicament.

In certain embodiments of the invention, the anti-proliferative drug or medicament used is commercially available. Some non limiting examples include carboplatin, cisplatin, docetaxel, paclitaxel, doxorubicin, HCl liposome injection, topotecan, hydrochloride, gemcitabine, cyclophosphamide, and etoposide or any combination thereof. Merely to illustrate, the anti-proliferative drug or medicament can be an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and/or a DNA repair inhibitor.

Chemotherapeutic agents as envisaged be the present invention may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitot- ic antimetabolites such as folic acid analogs (methotrexate); platinum coordination complexes (cisplatin, carboplatin, spiroplatin, iproplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors. Preferred dosages of the chemotherapeutic agents are consistent with currently prescribed dosages.

Most preferably, said anti-proliferative drug or medicament is selected from the group consisting of cisplatin, carboplatin, 5-fluorouracil, paclitaxel and docetaxel.

In another preferred embodiment said further anti-proliferative drug or medicament is to be administered before, during or after the administration of the pharmaceutical composition of the invention. Accordingly, the anti-proliferative drug or medicament may be administered within 1, 2, 3 or 4 weeks before or after the polyclonal antiserum.

As one example, the polyclonal antiserum may be administered within a chemotherapy schedule. The chemotherapy can be given in 3-4 week cycles or other schedules according to the treating physician and common clinical practice. Chemotherapy may continue for up to six cycles followed by the polyclonal antibody administration every twelve weeks for up to two years.

Also envisaged is a combined schedule of chemotherapy and antiserum administration wherein, for example, the polyclonal antiserum of the invention (or parts thereof as described above) is/are given by intravenous infusion over 20 minutes in a dose equal to or less than 2 mg during weeks, e.g. 1, 3, 5, 9, then every 8 weeks, followed by administration of a chemotherapeutic drug within, for example 5 days. The attending physician is readily in a position to deduce a corresponding treatment scheme in accordance with the (medical) needs of each individual patient to be treated by use of the polyclonal antibodies/polyclonal serum of this invention.

The figures show:

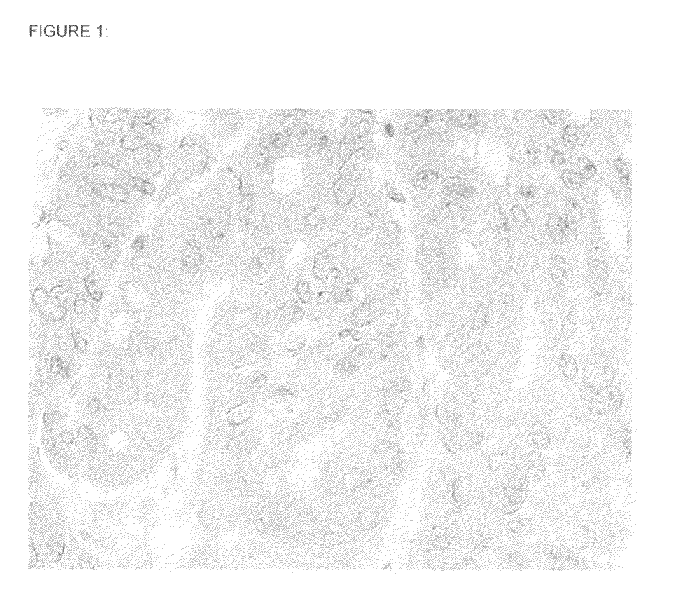

FIG. 1.—Colon adenocarcinoma from Checkerboard multi-tumor block (Dako), lot number 022145 stained with polyclonal antisera F(ab)′₂ fraction. Magnification: 40×.

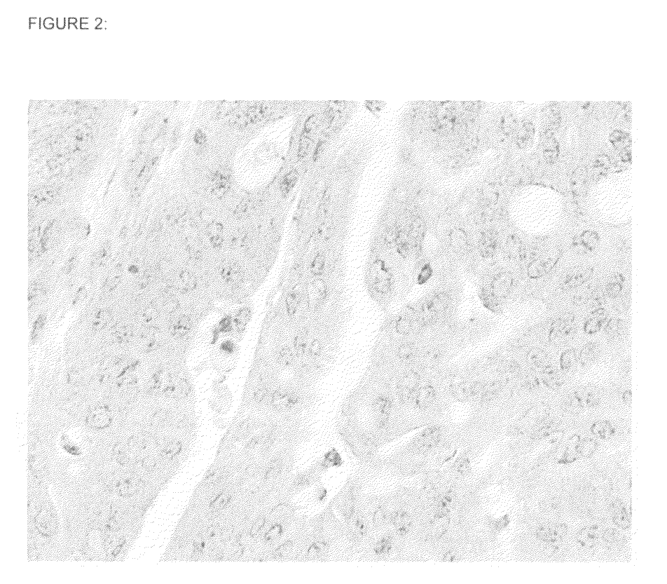

FIG. 2.—Colon adenocarcinoma from Checkerboard multi-tumor block (Dako), lot number 022145 stained with polyclonal antisera F(ab)′₂ fraction. Magnification: 40×

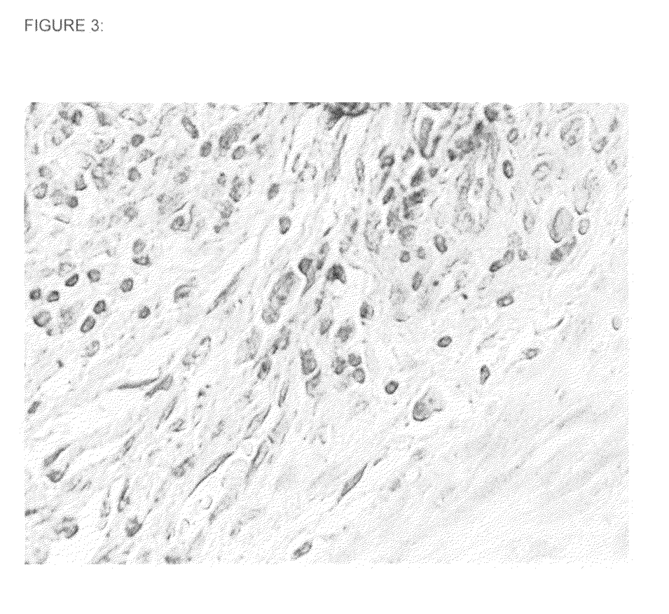

FIG. 3.—Gastric carcinoma from Checkerboard multi-tumor block (Dako), lot number 022145 stained with polyclonal antisera F(ab)′₂ fraction. Magnification: 40×.

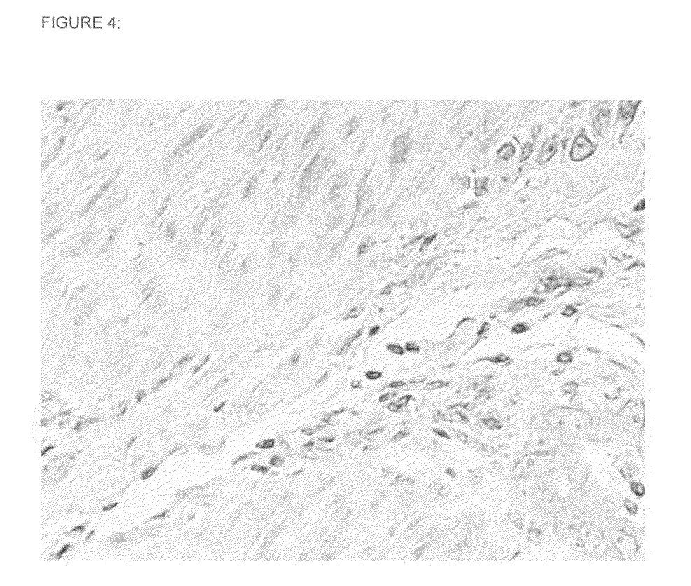

FIG. 4.—Gastric carcinoma from Checkerboard multi-tumor block (Dako), lot number 022145 stained with polyclonal antisera F(ab)′₂ fraction. Magnification: 40×

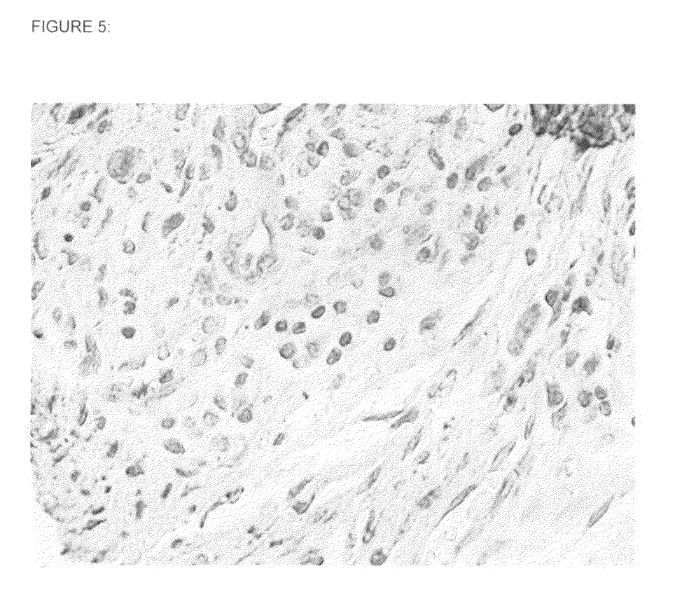

FIG. 5.—Gastric carcinoma from Checkerboard multi-tumor block (Dako), lot number 022145 stained with polyclonal antisera F(ab)′₂ fraction. Magnification: 40×.

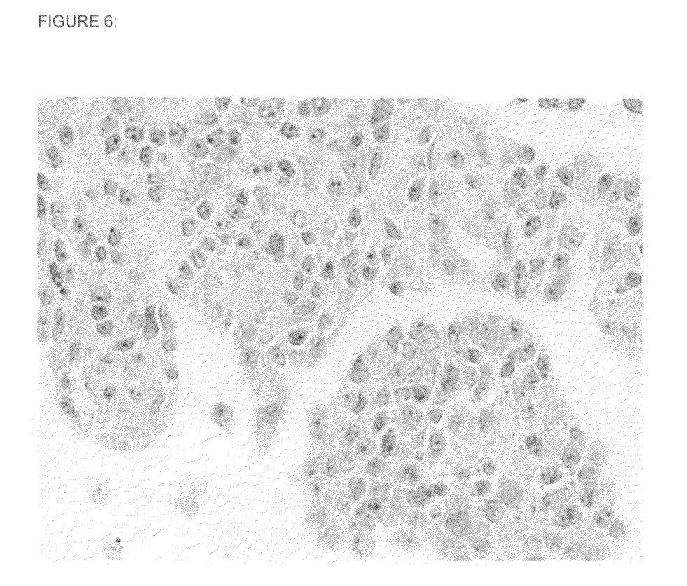

FIG. 6.—Breast carcinoma from Checkerboard multi-tumor block (Dako), lot number 022145 stained with polyclonal antisera F(ab)′₂ fraction. Magnification: 40×.

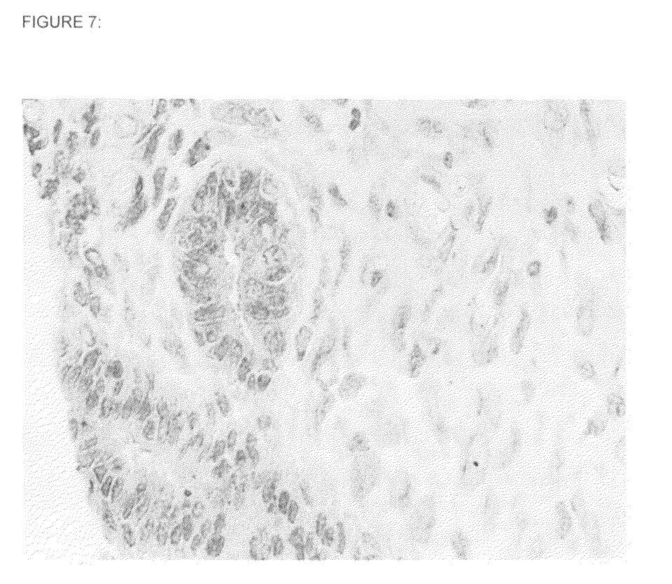

FIG. 7.—Breast carcinoma from Checkerboard multi-tumor block (Dako), lot number 022145 stained with polyclonal antisera F(ab)′₂ fraction. Magnification: 40×

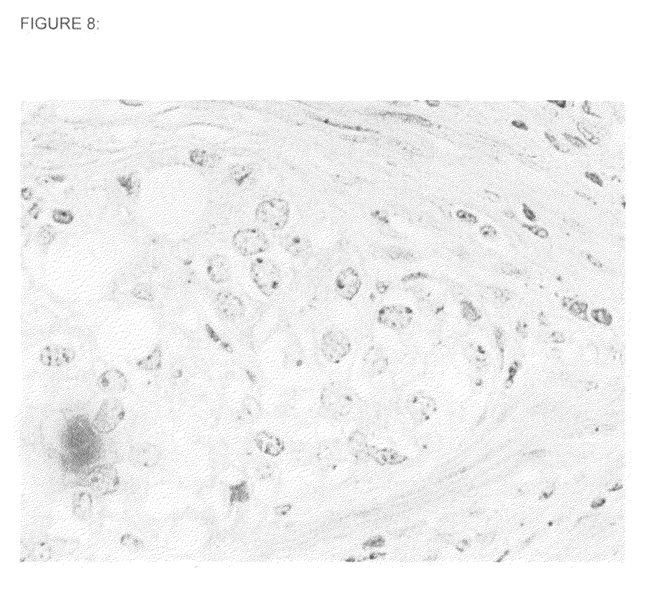

FIG. 8.—Prostate carcinoma from Checkerboard multi-tumor block (Dako), lot number 022145 stained with polyclonal antisera F(ab)′₂ fraction. Magnification: 40×.

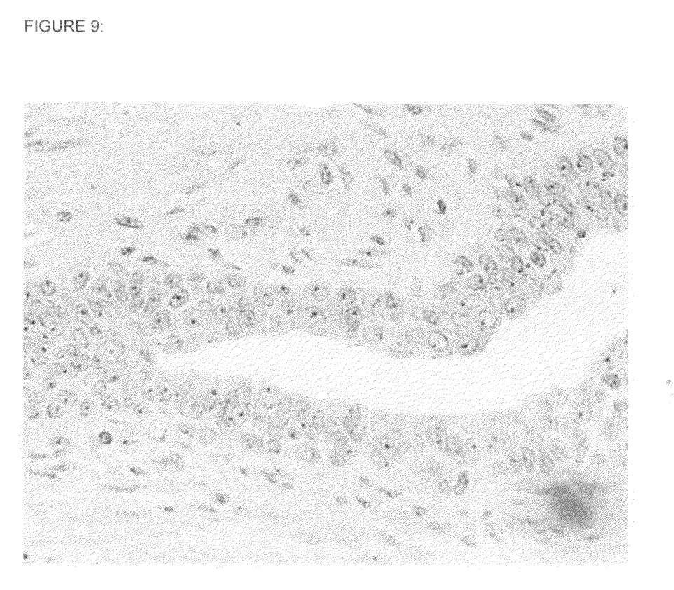

FIG. 9.—Prostate carcinoma from Checkerboard multi-tumor block (Dako), lot number 022145 stained with polyclonal antisera F(ab)′₂ fraction. Magnification: 40×.

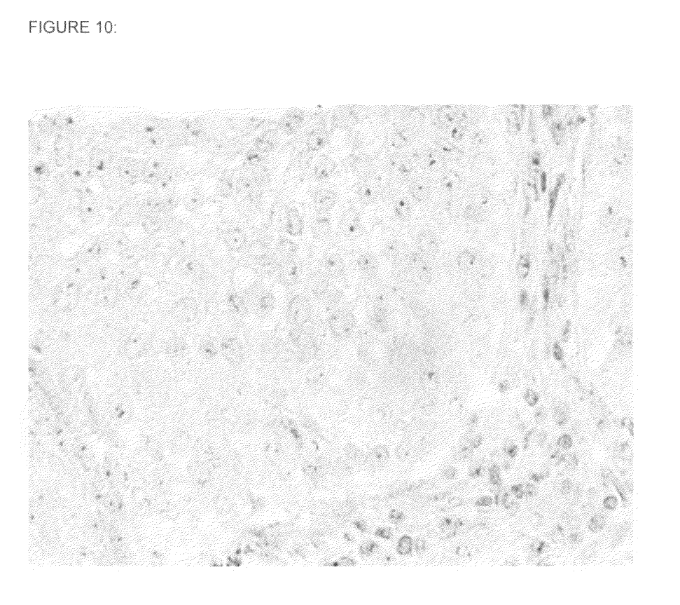

FIG. 10.—Lung adenocarcinoma from Checkerboard multi-tumor block (Dako), lot number 022145 stained with polyclonal antisera F(ab)′₂ fraction. Magnification: 40×.

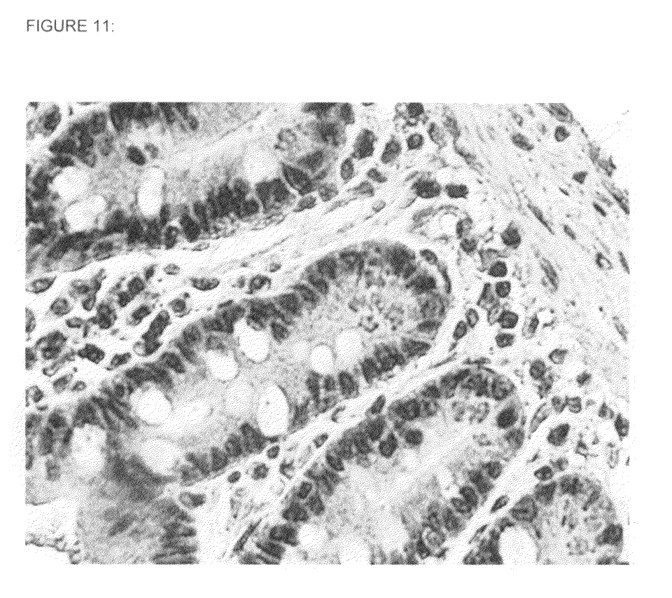

FIG. 11.—Small intestine normal tissue from Checkerboard multi-normal block (Dako), lot number 00121 stained with polyclonal antisera F(ab)′₂ fraction. Magnification: 40×.

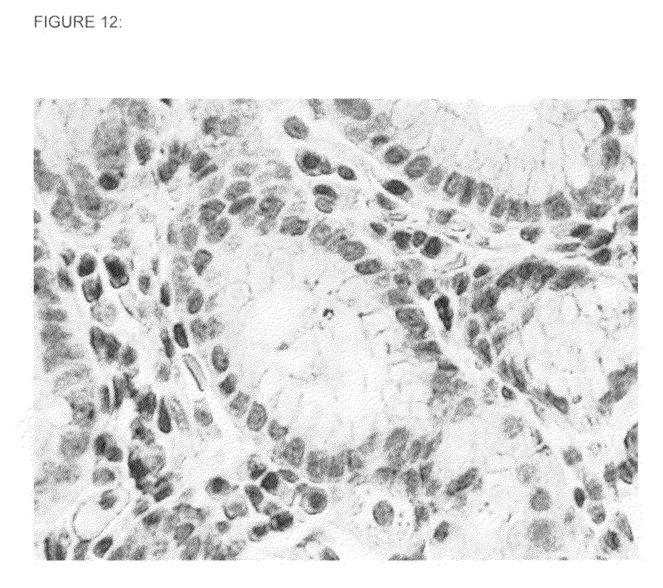

FIG. 12.—Stomach normal tissue from Checkerboard multi-normal block (Dako), lot number 00121 stained with polyclonal antisera F(ab)′₂ fraction. Magnification: 40×.

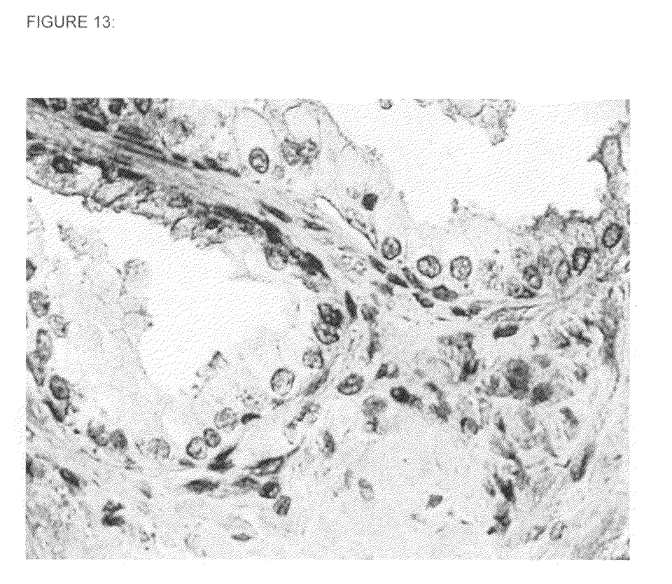

FIG. 13.—Prostate normal tissue from Checkerboard multi-normal block (Dako), lot number 00121 stained with polyclonal antisera F(ab)′₂ fraction. Magnification: 40×.

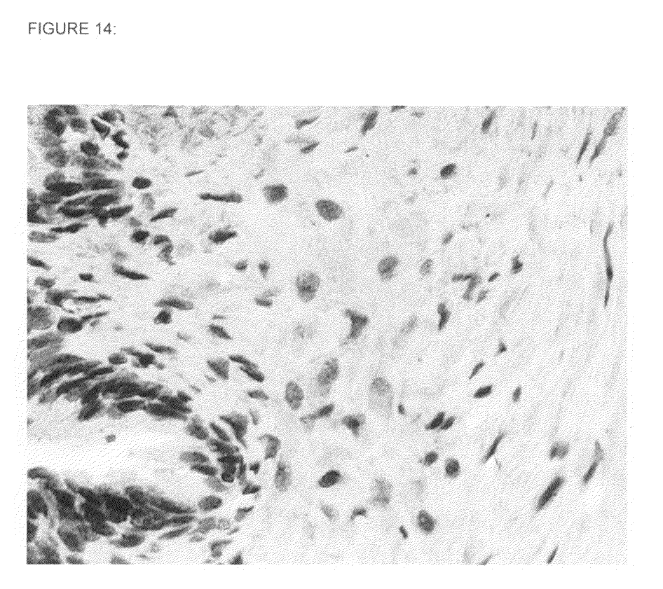

FIG. 14.—Prostate normal tissue from Checkerboard multi-normal block (Dako), lot number 00121 stained with polyclonal antisera F(ab)′₂ fraction. Magnification: 40×.

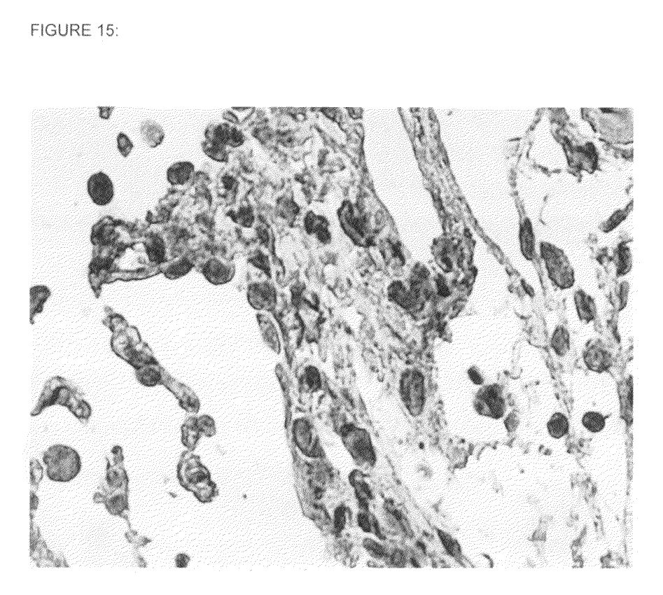

FIG. 15.—Lung normal tissue from Checkerboard multi-normal block (Dako), lot number 00121 stained with polyclonal antisera F(ab)′₂ fraction. Magnification: 40×.

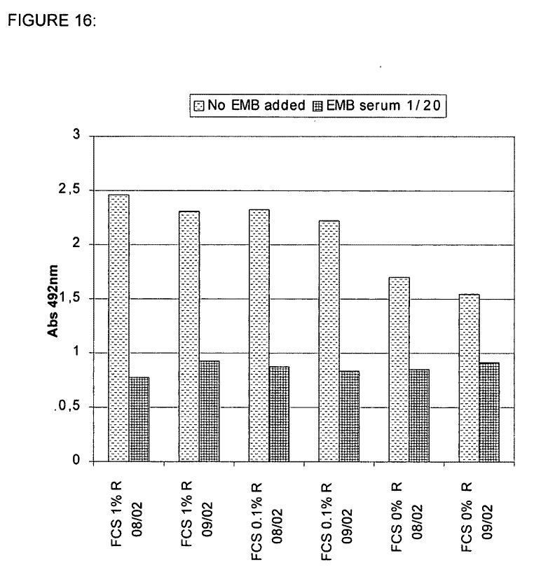

FIG. 16.—In vitro effect of polyclonal antiserum (lots 09/02 and 08/02) on HeLa cells. The average absorbance values obtained from a 96 well plate inoculated with HeLa cells and treated with a 1/20 dilution of polyclonal antiserum lots 09/02 and 08/02 in culture media is shown. After 24 hours, the MTS/PMS reagent was added to the plate and the absorbance was recorded after 4 hours of incubation.

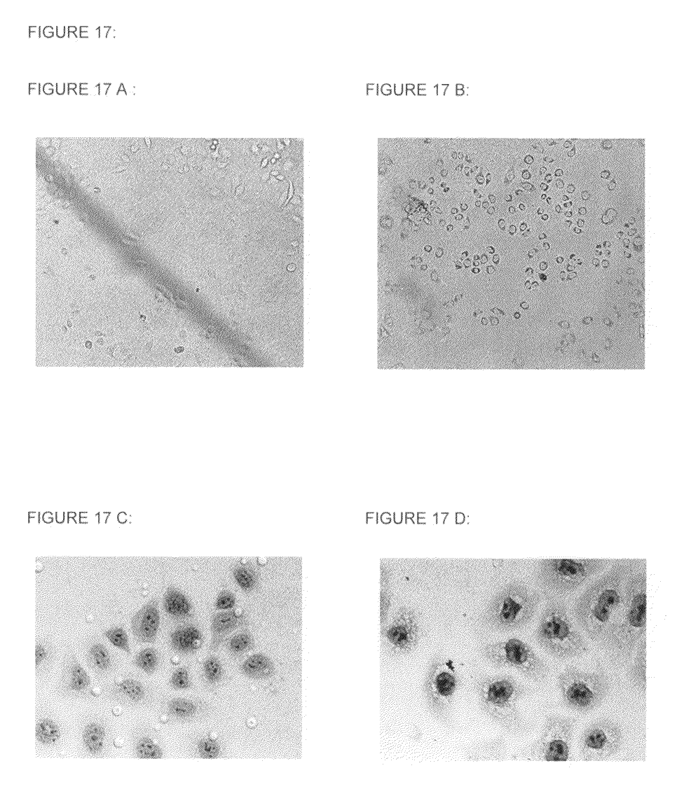

FIG. 17.—Photomicrographies of HeLa cells growing in MEM 1% FCS. In 17a cells were grown in MEM 1% FCS (10×). In 17b cells were exposed to APU sera lot R09/02 (diluted 1/20 in MEM 1% FCS) for 24 hours (10×). In 17c. HeLa cells in MEM 1% details (40×). In 17d. HeLa cells in MEM 1% with R09/02 (40×).

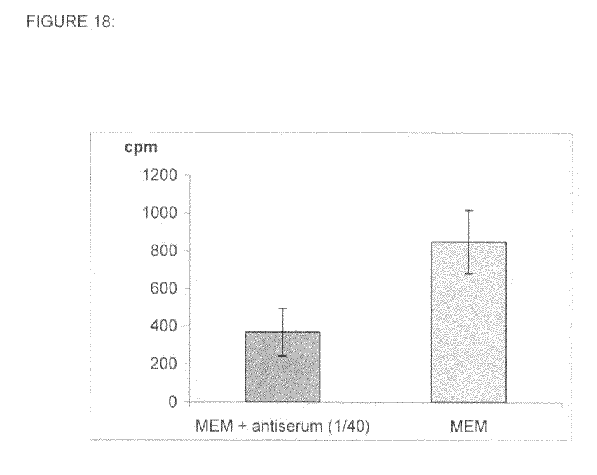

FIG. 18.—In vitro effect of polyclonal antiserum on HeLa cells in a H³-thymidine incorporation assay. The incorporation values obtained from a 96 well plate inoculated with HeLa cells and treated with a 1/40 dilution of polyclonal antiserum lot 0902 in culture media is shown. After 24 hours, complete MEM and H³-thymidine was added to the plate and the incorporation of H³-thymidine after 16 hours of incubation was recorded.

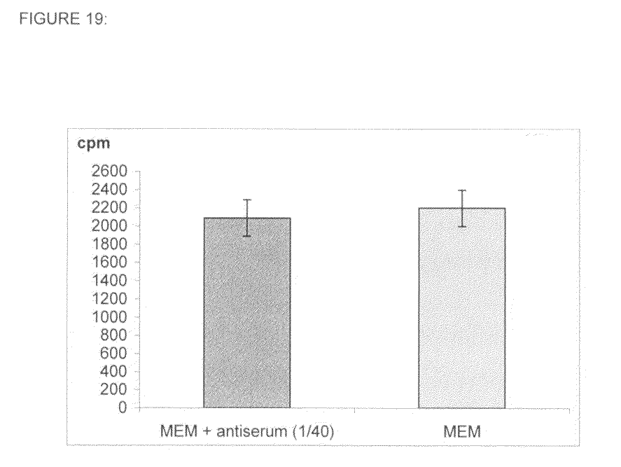

FIG. 19.—Growth inhibition of fibroblast cells by a polyclonal antiserum against universal tumor antigen. The incorporation values obtained from a 96 well plate inoculated with fibroblasts and treated with a 1/40 dilution of polyclonal antiserum lot 0902 in culture media are shown. As a control normal serum (Lot C0402) as used.

After 24 hours, complete MEM and H³-thymidine was added to the plate and the incorporation of H³-thymidine after 16 hours of incubation was recorded.

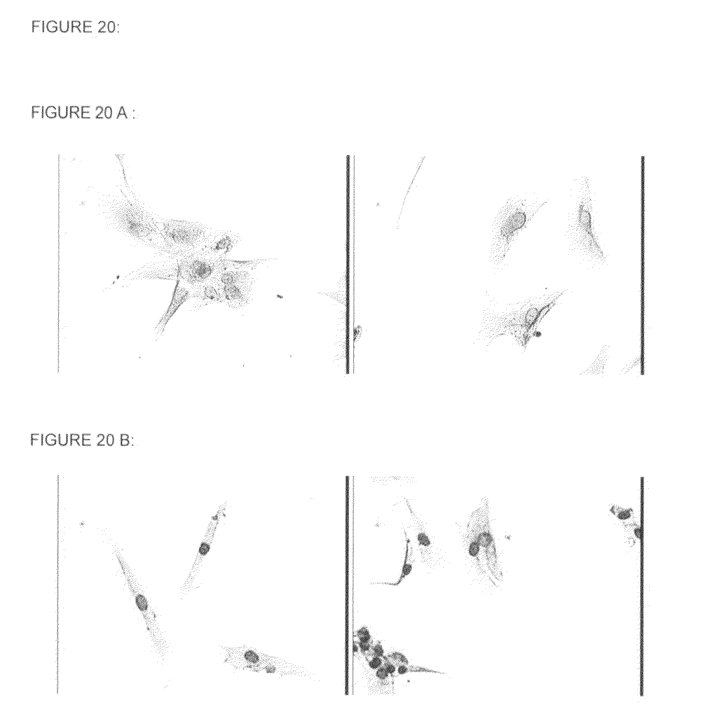

FIG. 20.—A: Immunohistochemistry stain of human fibroblast (CCD-986-Sk, ATCC) with a reactive serum. Fixed cells were incubated with the anti-idiotype serum. Reaction was developed with the DAKO LSAB 2 kit. Nucleus were stained with Mayer's Hematoxylin.

B: Immunohistochemistry stain of human fibroblast (CCD-986-Sk, ATCC) with control serum. Fixed cells were incubated with control serum. Reaction was developed with the DAKO LSAB 2 kit. Nucleus were stained with Mayer's Hematoxylin.

The invention is illustrated by the following examples:

### EXAMPLE 1

### Obtainment of Embryonic Cells

Five female and two male mice, 6 to 8 weeks old, of inbreed strain CBA/CaJ (Jackson Laboratory, USA, Stock Number 000654), were mated together in 20×30 cm cages for 12 hours, starting in the afternoon. Subsequently, pregnancy was established by vaginal plug detection and pregnant females were placed apart. This day was considered as day 1 of pregnancy.

On day 12 of pregnancy the mice were killed by cervical dislocation. Immediately after death, the mice were placed over a dissecting board and fixed facing up. The skin and peritoneal membrane were cut with a surgical scissors along the ventral midline from the groin to the chin.

The uterus was removed and transferred to a Petri dish with Versene solution (0.2 g Na₄EDTA in 1 l PBS). Embryos were separated from the embryonic sac with forceps and scissors and washed twice with Versene solution (0.2 g Na₄EDTA in 1 l PBS). Yolk sacs were discarded. From 12 vaginal plugs detected 7 were pregnant.

Embryos soaked in Versene solution (0.2 g Na₄EDTA in 1000 ml PBS [NaCl 137 mM, KCl 2.7 mM, Na₂HPO₄ 4.3 mM, KH₂PO₄ 1.4 mM, pH 7.3±0.1]) were homogenized mechanically with a tissue grinder in order to release the cells.

Homogenized embryos were centrifuged at 370×g for 20 min at 4° C. and washed twice with Medium 199 (Sigma Aldrich, Product #M4530) with 1 ml per 2 pregnant mice. The material was allowed to settle down for 1 min. Free cells were aspirated with a sterile syringe (25 G ⅝ needle) to avoid tissue clumps.

Isolated cells were diluted at least 1/100 in Versene solution (0.2 g Na4EDTA in 1 l PBS) and counted in a Neubauer improved counting chamber.

### EXAMPLE 2

### Obtainment of Embryo-Specific Spleen Cell Enriched Fractions

The embryonic cells as described herein above were centrifuged at 370×g for 20 min at 4° C. Supernatant was discarded and the cells were mixed with complete Freund's adjuvant (CFA) in a concentration of 10×10⁶ cells in 0.1 ml CFA/animal. Each dose was prepared separately. 6 to 8 weeks old intact male mice of inbreed strain CBA/CaJ (Jackson Laboratory, USA, Stock Number 000654) were immunized by subcutaneous injection with 0.1 ml emulsion obtained by the above step of mixing the cells with Freund's adjuvant. To obtain 10 ml reactive serum about 70 male mice were required.

Five day after inoculation, mice were killed by cervical dislocation and the spleens were dissected.

Spleens from immunized mice obtained by the above step were collected in a Petri dish containing Medium 199 (Sigma Aldrich, Product # M4530) and cut into small pieces with scissors. Subsequently, small pieces were ground with a Potter-Elehjen tissue grinder until a single-cell suspension was obtained. Cell-clumps were disrupted by repeated passages through a 19-G needle with the aid of a syringe. The spleen cell suspension was filtered through a 200 μm nylon mesh, transferred to a 15 ml polypropylene conical tube and centrifuged at 200×g for 10 min at 4° C. The supernatant was discarded. 5 ml of an ammonium chloride solution (NH₄Cl 0.15 M, KHCO₃ 1 mM, Na₂EDTA 0.1 mM, pH 7.3±0.1) per processed spleen was added to the cellular pellet, gently mixed and incubated during 5 min at room temperature in order to lyse contaminating red blood cells. After incubation, the mixture was centrifuged at 200×g for 10 min at 4° C. The supernatant was discarded. Remaining spleen was washed with Medium 199 (Sigma Aldrich, Product # M4530) and suspended in 20 ml of Medium 199.

### EXAMPLE 3

### Antiserum Preparation

The spleen cells as described herein above were centrifuged at 200×g for 10 min at 4° C., the supernatant was discarded and the cells were mixed with complete Freund's adjuvant (CFA) in a ratio of 10×10⁶ cells per 0.1 ml CFA/animal. Each dose was prepared separately. Intact male mice were immunized by intraperitoneal injection with 0.1 ml emulsion obtained as described herein above. In order to obtain 10 ml serum approx. 50 male mice were inoculated.

Four days after the inoculation the mice were killed by cervical dislocation, placed over a dissecting board and fixed facing up. Skin and peritoneal membrane were cut with surgical scissors along the ventral midline from the groin to the chin. The ribs were cut and the thorax cavity opened. The lungs were put aside carefully without removing them and the heart was cut out. The blood was collected from the thoracic cavity and refrigerated at 4° C. until clot. Subsequently the blood was centrifuged at 370×g for 20 min at 4° C. and the thereby obtained serum was pooled together. Afterwards a heat inactivation at 56° C. was carried out for 15-20 min.

Undesirable cross reactivity with normal tissue was avoided by incubating with freshly isolated organ cells from normal mice. To this end 6 to 8 weeks old, CBA/CaJ mice (Jackson Laboratory, USA, Stock Number 000654) were killed by cervical dislocation, placed over a dissecting board and fixed facing up. Skin and peritoneal membrane were cut with surgical scissors along the ventral midline from the groin to the chin. Heart, liver and kidney cells were dissected and the organs were place into a Petri dish and washed with Medium 199 (Sigma Aldrich, Product # M4530). The heart was pressed with forceps in order to extract blood and fat was separated from the organs.

The organs were then cut into small pieces with scissors and placed into a 15 ml polypropylene conical tube with a Pasteur pipette. Subsequently, the pieces were centrifuged at 370×g for 10 min at 4° C. and washed twice with Medium 1999. The supernatant was discarded. The cells were incubated for 29 to 25 min at room temperature with blood obtained as described herein above. After incubation, the mixture was centrifuged at 370×g for 290 min at 4° C. to eliminate coarse solid material. The so obtained supernatant was subjected to a final clarification by centrifuging at 3500×g at 4° C. during 10 min and stored at −70° C.

A further protocol for the preparation of antiserum comprises the following steps:

The spleen cells as described herein above were centrifuged at 200×g for 10 min at 4° C., the supernatant was discarded and the cells were mixed with complete Freund's adjuvant (CFA) in a ratio of 10×10⁶ cells per 0.1 ml CFA/animal. Each dose was prepared separately. Intact male mice were immunized by intraperitoneal injection with 0.1 ml emulsion obtained as described herein above. In order to obtain 10 ml serum approx. 50 male mice were inoculated. Four days after the first immunization the mice were boosted with freshly prepared cells as described herein above. Male mice were boosted by intraperitoneal injection with 0.1 ml emulsion obtained as described herein above.

Four days after the last inoculation the mice were killed by cervical dislocation, placed over a dissecting board and fixed facing up. Skin and peritoneal membrane were cut with surgical scissors along the ventral midline from the groin to the chin. The ribs were cut and the thorax cavity opened. The lungs were put aside carefully without removing them and the heart was cut out. The blood was collected from the thoracic cavity and refrigerated at 4° C. until clot. Subsequently the blood was centrifuged at 370×g for 20 min at 4° C. and the thereby obtained serum was pooled together. Afterwards a heat inactivation at 56° C. was carried out for 15-20 min.

Undesirable cross reactivity with normal tissue was avoided by incubating with freshly isolated organ cells from normal mice. To this end 6 to 8 weeks old, CBA/CaJ mice (Jackson Laboratory, USA, Stock Number 000654) were killed by cervical dislocation, placed over a dissecting board and fixed facing up. Skin and peritoneal membrane were cut with surgical scissors along the ventral midline from the groin to the chin. Heart, liver and kidney cells were dissected and the organs were place into a Petri dish and washed with Medium 199 (Sigma Aldrich, Product # M4530). The heart was pressed with forceps in order to extract blood and fat was separated from the organs.

The organs were then cut into small pieces with scissors and placed into a 15 ml polypropylene conical tube with a Pasteur pipette. Subsequently, the pieces were centrifuged at 370×g for 10 min at 4° C. and washed twice with Medium 1999. The supernatant was discarded. The cells were incubated for 29 to 25 min at room temperature with blood obtained as described herein above. After incubation, the mixture was centrifuged at 370×g for 290 min at 4° C. to eliminate coarse solid material. The so obtained supernatant was subjected to a final clarification by centrifuging at 3500×g at 4° C. during 10 min and stored at −70° C.

### EXAMPLE 4

### Affinity Purification of Polyclonal Antibodies

Starting from the herein above described polyclonal antiserum an IgG purification was carried out by G protein affinity chromatography using a 1 mL HiTrap HiTrap™ column (Amersham Bioscience, USA) according to manufacturer instructions. 2 mL of the polyclonal antiserum were diluted to 6 ml with binding buffer (20 mM sodium phosphate, pH 7.0), and centrifuged at 12,000 g for 5 minutes. The supernatant was applied to a binding-buffer equilibrated column at a flow rate of 1 mL/min. The column was washed with 20 mL of binding-buffer until AbS_{280 nm}<0.01. Adsorbed IgG were eluted with 5 mL of elution buffer (0.1 M glycine-HCl, pH 2.7). 500 μl fractions were collected in tubes containing 50 μl of 1 M Tris-HCl, pH 9.0 in order to preserve the activity of acid labile IgGs. Eluted IgG was followed by UV monitoring at 280 nm. Usually, IgG eluted within the first 3 mL. Fractions showing Abs 280 nm>0.2 were pooled and dialyzed against PBS (NaCl 137 mM, KCl 2.7 mM, Na₂HPO₄ 4.3 mM, KH₂PO₄ 1.4 mM, pH 7.3±0.1) and stored at −20° C.

Purified IgGs, obtained by the steps described herein above were digested with pepsin, using agarose immobilized pepsin (ImmunoPure® F(ab′)₂ preparation kit, Cat # 44888, Pierce, USA), according to the manufacturer's recommendations with some modifications. In particular, an IgG solution and agarose immobilized pepsin were equilibrated with acetic acid 20 mM, pH 2.8, and mixed. Digestion was carried out at 37° C. with gentle agitation. After 2 hours incubation, the supernatant containing F(ab′)₂ fragments was separated and 1 M Tris was added to increase pH near neutrality. The F(ab′)₂ containing fraction was dialyzed with PBS and stored at −20° C. About 90% conversion of IgG to F(ab′)₂ was achieved as pointed out by SDS-PAGE analysis.

### EXAMPLE 5

### Quality Test and Determination of the Protein Concentration of the Affinity Purified Polyclonal Antibodies

The quality of the IgG and F(ab′)₂ preparations was examined by polyacrylamide gel electrophoresis in presence of sodium dodecyl sulfate (SDS-PAGE) according to Laemmli, 1970. Protein concentration was determined by the bicinchoninic acid method according to Smith, 1985 employing BCA™ Protein Assay Kit (cat #23227, Pierce, USA).

### EXAMPLE 6

### Tumour Specific Immunostaining

Formalin-fixed paraffin-embedded tissue sections were immunostained for tumor-specific recognition. Checkerboard multi-normal block slides, (Catalog No. T1065) and Checkerboard multi-tumor block slides (Catalog No. T1064) were purchased from Dako, USA.

Tissue sections were deparaffinized, rehydrated and subjected to antigen retrieval procedure using Target Retrieval Solution (Dako, USA, Catalog No. S1699) in a boiling water bath for 20 min. After slowly cooling, tissue sections were blocked for non-specific binding by incubation with 1% bovine seroalbumin (BSA Sigma, USA, Catalog No. A 7030) solution in phosphate-buffered saline (PBS [NaCl 137 mM, KCl 2.7 mM, Na₂HPO₄ 4.3 mM, KH₂PO₄ 1.4 mM, pH 7.3±0.1]) for 30 minutes at room temperature. After washing, polyclonal IgG (F(ab′)₂ fragment) 40 μg/mL solution in PBS containing 1% BSA was added to each tissue section (50 μL approximately) and incubated overnight in a humid chamber at 4° C. and then washed. Any intrinsic peroxidase activity was blocked incubating with Ready-to-use Peroxidase Blocking Reagent (Dako, USA, Catalog No. S2001) following manufacturer guidelines. After extensive washing, tumor-specific reaction was detected using HRP-LSAB 2 system (Dako, USA, Catalog No. K0675) and Liquid DAB Substrate-Chromogen System (Dako, USA, Catalog No. K3465) according to manufacturer directions.

Counterstaining with Haematoxylin-eosin was done prior to microscopic analysis.

### EXAMPLE 7

### Effect of the Polyclonal Antiserum on Hela Cell Cultures in a Cell Proliferation Assay

In order to quantify the cytotoxic effect of the polyclonal antiserum on HeLa cells a colorimetric method using a tetrazolium salt (MTS) that is reduced to give coloured formazans (Rode, 2004), as a subtrate for mitochondrial activity has been used. HeLa cells (ATCC No. CCL-2) were routinely maintained in Minimal Essential Medium (MEM, Sigma) containing L-glutamine (2 mM), supplemented with 5% fetal calf serum (FCS, Bio Whitaker) and 40 μg/mL gentamicine (Sigma, USA). Twenty-five square centimetres or 75 cm² tissue culture flasks (Nunc, USA) containing the cultures were incubated in a humidified, 5% CO₂ incubator at 37° C. When required, HeLa cells were sub cultured by standard trypsinization procedures (Morgan and Darling, 1993). The medium was removed by aspiration, cells were washed with FCS free medium and incubated with 3 mL of trypsin-EDTA solution (1% trypsin, 1 mM EDTA, pH 8.0 in PBS, Sigma) for five minutes at 37° C. Detached cells were washed with complete medium (5 mL per 25 cm² of original culture), and resuspended at a concentration of 1×10⁵ cells/mL in complete medium. Only cells with viability greater than 95%, as determined by Trypan Blue dye exclusion (2), were used for testing.

The cell proliferation assays were performed in 96-well tissue culture plates (Greiner, Germany). 100 μL of HeLa cell suspension (1×10⁵ cells/mL) were placed into the plate wells using a multichannel pipette as outlined in Table 1. Microplates were incubated in a humidified, 5% CO2 incubator at 37° C. for 24 hours in order to allow cells to attach. Medium was removed by aspiration and cells were washed twice with FCS free MEM medium. The polyclonal antiserum was sterilised by filtration and appropriately diluted in selected media (100 μL per well) and subsequently added, in at least four replicates, into HeLa cell containing microplates using a multichannel pipette as described in Table 1. Microplates were incubated in a humidified, 5% CO2 incubator at 37° C. for 24, 48 or 72 hours before adding the MTS/PMS detection reagent.

Polyclonal antiserum lots R 02/02, R 08/02 and R 09/02 were selected for cell proliferation assays. Sera were diluted 1/20 in MEM media containing 1% FCS, 0.1% FCS or FCS free medium (0% FCS) and sterilized by 0.22 μm membrane filtration. Dilutions were freshly prepared.

TABLE 1
Outline of the cell proliferation assay.

| | 1 | 2 | 3 | 4 | 5 | 6 | 7 | 8 | 9 | 10 | 11 | 12 |
|---|---|---|---|---|---|---|---|---|---|---|---|---|
| A | Only | HeLa + | HeLa + | HeLa + | HeLa + | HeLa + | Only | HeLa + | HeLa + | HeLa + | HeLa + | HeLa + |
| B | medium | MEM | C01/02 | C03/02 | R08/02 | R09/02 | medium | MEM | C01/02 | C03/02 | R08/02 | R09/02 |
| C | MEM | 1% | ( 1/20) | ( 1/20) | ( 1/20) | ( 1/20) | MEM | 0.1% | ( 1/20) | ( 1/20) | ( 1/20) | ( 1/20) |
| D | 1% | FCS | in | in | in | in | 0.1% | FCS | in | in | in | in |
| E | FCS | | MEM | MEM | MEM | MEM | FCS | | MEM | MEM | MEM | MEM |
| F | | | 1% | 1% | 1% | 1% | | | 0.1% | 0.1% | 0.1% | 0.1% |
| G | | | FCS | FCS | FCS | FCS | | | FCS | FCS | FCS | FCS |
| H | | | | | | | | | | | | |

Ninety-six well tissue plates were inoculated with 10⁴ HeLa cells per well (except for columns 1 and 7), cells were allowed to attach and then incubated with antisera for 24, 48 or 72 hours. Polyclonal antisera effect on cell viability was measured by the MTS/PMS assay.
R: APU sera;
C: mouse sera.

The Promega CellTiter 96® AQueous Non-Radioactive assay (Promega, USA Catalog No. G5421; Barltrop, 1991; Cory, 1991) was employed to determine the effect of the polyclonal serum addition to HeLa cell cultures. Solutions were prepared according to the manufacturer's instructions. Stock MTS (Promega, USA) and PMS (ICN, USA) were dissolved in DPBS (SIGMA) at a concentration of 2.0 mg/mL and 0.92 mg/mL, respectively. Solutions were filtered through 0.22 μm sterile membrane (Sartorius, Germany, Catalog No. 16534) and stored in light-protected tubes at −20° C. MTS and PMS detection reagents were mixed immediately before use in sterile conditions, at a ratio of 20:1 (MTS: PMS), and added to the cell culture at a ratio of 1:5 (20 μL reagent each 100 μL medium). After MTS/PMS addition, microplates were incubated for 4 hours in a humidified, 5% CO₂ incubator at 37° C. During incubation, colour development was monitored at 492 nm every hour using a computer-connected Multiskan MS microplate reader (Thermo Labsystems).

The results obtained showed a decrease in metabolic activity of cells treated with the polyclonal antiserum compared to cells growing in complete MEM media (see FIG. 16). This effect could be explained by a lower number of cells or by lower metabolic activity of the cells when treated with APU sera or could be the sum of both effects. However when examined by microscopy, polyclonal antisera treated cells shown clear sign of stress. An apparent cytoplasmic vacuolation (FIG. 17d) and membrane changes (FIG. 17b) could be appreciated in HeLa cells cultures exposed to the polyclonal antiserum.

### EXAMPLE 8

### Effect of the Polyclonal Antiserum on Hela Cell Cultures in a H³-thymidine Cell Growth Assay

In order to quantify the cytotoxic effect of the polyclonal antiserum on HeLa cells a method based on the incorporation of H³ thymidine during DNA synthesis has been used.

HeLa cells (ATCC No. CCL-2) were grown in Minimal Essential Medium (Eagle) containing L-glutamine (2 mM) and Earle's BSS adjusted to contain 2 g/L sodium bicarbonate, 0.1 mM non-essential amino acids, 1.0 mM sodium pyruvate, penicillin (100 U/ml), streptomycin (100 g/l), supplemented with 10% fetal bovine serum (complete MEM medium) to obtain a monolayer of adherent cells as 37° C. in 5% CO₂. The cells were detached by treatment with trypsin solution (=0.25% trypsin, 0.53 mM EDTA). The cells were resuspended at 3200 cells/ml in complete medium, dispensed (200 μl/well) in 96-well plates and incubated during 5 days at 37° C. in 5% CO₂.

At day 5 the medium was removed and different dilutions (60, 30, 15, 7.5, 3.25 μg/ml) of Protein-G-purified immunoglobulins from polyclonal antiserum (Lot 0902) in culture medium without fetal bovine serum (200 μl/well, by quadruplicate) were added and incubated during 24 h at 37° C. in 5% CO₂. The same dilutions of Protein G-purified immunoglobulins from normal serum (Lot C0402) were used as a control. Subsequently, the supernatant was removed, complete medium (200 μl/well) was added and the cells were pulsed with 1 μCi/well of H³-thymidine for 16 h at 37° C. in 5% CO₂.

Subsequently, the cells were harvested and H³-thymidine incorporation was assessed using a 96-well plate beta-counter. The mean c.p.m. −/+standard deviation (SD) was calculated for each quadruplicate.

The results obtained showed a decrease of H³-thymidine incorporation in the case of cells treated with purified immunoglobulins form polyclonal antiserum compared with the controls. This effect was dose dependent (see FIG. 18).

### EXAMPLE 9

### Growth Inhibition of Fibroblast Cells by a Polyclonal Antiserum Against Universal Tumor Antigen

Fibroblasts (ATCC No. CCD-986Sk) were grown in Iscove's Modified Dulbecco's Medium with 4 mM L-glutamine adjusted to contain 1.5 g/L sodium bicarbonate, 90%, fetal bovine serum, 10%, to obtain a monolayer of adherent cells at 37° C. in 5% CO₂.

Subsequently, the cells were detached by treatment with trypsin solution (0.25% trypsin, 0.53 mM EDTA).

Cells were resuspended at 3200 cells/ml in complete medium, dispensed (200 μl/well) in 96-well plates and incubated for a period of 5 days at 37° C. in 5% CO₂. At day 5 the medium was removed and different dilutions (60, 30, 15, 7.5, 3.25 μg/ml) of Protein-G-purified immunoglobulins form polyclonal antiserum (Lot 0902) in culture medium without fetal bovine serum (200 μl/well, by quadruplicate) were added and incubated for a period of 24 h at 37° C. in 5% CO₂. The same dilutions of Protein G-purified immunoglobulins from normal serum (Lot C0402) were used as a control. Subsequently, the supernatant was removed, complete medium (200 μl/well) was added and the cells were pulsed with 1 μCi/well of H³-thymidine for 16 h at 37° C. in 5% CO₂.

Finally, the cells were harvested and H³-thymidine incorporation was assessed using a 96-well plate beta-counter. The mean c.p.m. −/+standard deviation (SD) was calculated for each quadruplicate.

The results obtained did not show a difference between H3-thymidine incorporation in the case of cells treated with purified immunoglobulins from polyclonal antiserum compared with the controls (see FIG. 19).

### EXAMPLE 10

### Immunochemistry Stain of Human Fibroblast

An immunochemistry stain of human fibroblast (ATCC No. CCD-986Sk) with reactive or control serum is shown in FIG. 20. Fixed cells were incubated with the anti-idiotype serum, the reaction was developed with the DAKO LSAB 2 kit and then nuclei were stained with Mayer's Hematoxylin (see FIG. 20A); or the fixed cells were incubated with control serum, the reaction was developed with the DAKO LSAB 2 kit and the nuclei were stained with Mayer's Hematoxylin (see FIG. 20B). No specific plasma membrane stain was observed neither when the cells were incubated with the anti-idiotypic serum nor with the control one. Background stain was observed for the nucleus as it could be expected when working with polyclonal antiserum in both situations.

### CITED REFERENCES

Abelev, 1971, Adv. Cancer Res., 14: 295-357.
American Cancer Society, 2003, Cancer Facts and Figures.
Arnon, et al., 1982, Cancer Surveys, 1: 429-449.
Barltrop et al., 1991, Bioorg. & Med. Chem. Lett. 1: 611.
Brennan et al., 1985, Science, 229 (4708): 81-3.
Cory et al., 1991, Cancer Comm. 3: 207-12.
Cumber et al., 1985, Meth in Enzymol, 12: 207-225.
Davol et al., 2004, Clin Prostate Cancer. 3(2): 112-21.
Eckelman et al., 1980, Cancer Res., 40: 3036-42.
Fanger et al., 1991, Trends Biotechnol. 9(11): 375-80.
Flechner, 1973, European Journal of Cancer, 9: 741-745.
Ghose et al., 1972, British Medical Journal, 3: 495-499.
Goldenberg et al., 1993, Immunol Today, 14(1): 5-7.
Hamann et al., 2002, Bioconjug Chem, 3: 40.
Harlow and Lane, 1988, Antibodies: A laboratory manual, Cold Spring Harbor Press
Harris, 2004, Lancet Oncol., 5(5): 292-302.
Hurwitz et al., 1975, Cancer Research, 35: 1175-1181.
Kass et al., 2002, Cancer Res., 62(17): 5049-5057.
Kimball, 1993, Biology, William C. Brown ed.
Kipriyanov et al., 2004, Curr Opin Drug Discov Devel. 7(2): 233-42.
Laemmli, 1970, Nature 227(5259): 680-5.
Liu Yuanfang et al., 1991, IUPAC report, 427-462.
Morgan and Darling, 1993, Animal Cell Culture; BIOS Scientific Publishers Ltd ed.
Mizejewski et al., 2003, Mol Cancer Ther., 2: 1243-1255.
Okaji et al., 2004, Cancer Sci., 95: 85-90.
Osawa et al., 1982, Cancer Surveys, 1: 373-388.
Remington's Pharmaceutical Sciences, 1990, 18.sup.th Edition, ed. A. Gennaro, Mack Publishing Co., Easton, Pa.
Rode et al., 2004, Apoptosis, Cell Death, and Cell Proliferation Manual. 3^{rd} edition Roche Applied Science.
Scappaticci et al., 2003, Vaccine, 21: 2667-77.
Smith et al., 1985, Anal. Biochem., 150(1): 76-85.
Stan, 1999, Cancer Res., 59: 115-121.
Stern, 2004, Facing Cancer, MacGraw-Hill ed.
Szekerke et al., 1972, Neoplasm, 19: 211-215.
Tatarinov, 1965, Vop. Khim. SSR, 11: 20-24.
Thorpe et al., 1982, Monoclonal Antibody-Toxin Conjugates: Aiming the Magic Bullet, 168.
Van Spriel, et al., 2000, Immunol Today; 21: 391-6.
Waldmann, 1991, Science, 252: 1657.
Waldmann, 1988, Monoclonal antibody therapy, Vol. 45, Karger ed.

1. A pharmaceutical composition comprising a polyclonal antiserum obtained by the following steps:
(i) eliciting an in vivo humoral response against embryonic tissue in a non-human vertebrate, whereby said embryonic tissue is of the same strain (in the description: “the same genetic line”) as the non-human vertebrate;
(ii) recovering from the immunized non-human animal spleen and isolating from said spleen individual spleen cells/lymphocytes;
(iii) eliciting a second in vivo humoral response to the isolated spleen cells/lymphocytes suspension obtained in step (ii) in a further non-human animal of the same strain (genetic line) as the non-human animal of step (i); and
(iv) isolating the desired polyclonal antiserum from said animal.

2. (canceled)

3. The pharmaceutical composition of claim 1, wherein said non-human animal to be immunized is a mouse.

4. The pharmaceutical composition of claims 1, wherein said mouse is a mouse of the CBA strain.

5. The pharmaceutical composition of claim 1, wherein said polyclonal antiserum is a purified polyclonal antiserum.

6. The pharmaceutical composition of claim 1, wherein said polyclonal antiserum comprises a fraction of said antiserum.

7. The pharmaceutical composition of claim 6, wherein said fraction of said polyclonal antiserum is an IgG fraction or is or comprises a F(ab)₂ or Fab fragment fraction.

8. The pharmaceutical composition of claim 7, wherein the polyclonal antibody or fragment thereof is conjugated to a therapeutic agent.

9. The pharmaceutical composition of claim 7, wherein the therapeutic agent is selected from the group consisting of etoposide, cyclophosphamide, doxorubicin, calicheamicin, ricin, abrin, or a radionuclide.

10. A method of treatment and/or amelioration of cancer in a subject, said method comprising the following step:
administering to a subject in need of such a treatment or such an amelioration a pharmaceutically active amount of a pharmaceutical composition of claim 1.

11. The method of claim 10, wherein said subject is a human patient.

12. The method of claim 10, whereby said cancer is a cancer of the head and neck, a cancer of the lung, a neoplasm of the mediastinum, a cancer of the gastrointestinal tract, a cancer of the genitourinary system, a cancer of the testis, a gynecologic cancer, a cancer of the breast, a cancer of the endocrine system, a sarcoma of the soft tissues and bone, a malignant mesothelioma, a cancer of the skin, a malignant melanoma, a neoplasm of the central nervous system, a cancer of childhood, a lymphoma, leukaemia or an AIDS-related malignancy.

13. The method of claim 12, whereby said cancer is cancer of the uterus.

14. The method of claim 10, whereby said polyclonal serum or said pharmaceutical composition is to be administered to a subject in need of treatment in combination with a further anti-proliferative drug or medicament.

15. The method of claim 14, wherein said further anti-proliferative drug or medicament is to be administered before, during or after the administration of the pharmaceutical composition as defined in any one of claims 1 or 3 to 9.

16. The method of claim 14, whereby said anti-proliferative drug or medicament is selected from the group consisting of cisplatin, carboplatin, 5-fluorouracil, paclitaxel and docetaxel.
